(19) United States
(12) Patent Application Publication
(10) Pub. No.: US 2003/0165446 A1
(43) Pub. Date: Sep. 4, 2003

(54) Divalent manganese salts for reducing odour resulting from bacterial decomposition of human perspiration

(76) Inventors: Christophe Courbiere, Paris (FR); Serge Forestier, Claye Souilly (FR)

Correspondence Address: FINNEGAN, HENDERSON, FARABOW, GARRETT & DUNNER, LLP, 1300 I STREET, NW, WASHINGTON, DC, 20005, US

(21) Appl. No.: 10/257,756

(22) PCT Filed: Apr. 11, 2001
(86) PCT No.: PCT/FR01/01111

(51) Int. Cl. A61K 7/32

(52) U.S. Cl. 424/65

(57) ABSTRACT

The invention concerns, for the preparation of cosmetic compositions, the use of divalent manganese salts as agents for reducing odour resulting from bacterial decomposition of human perspiration. The invention also concerns a method for reducing human body odours resulting from their bacterial decomposition and in particular axillary odours, foot odours and scalp odours.

A subject matter of the present invention is the use, in the preparation of cosmetic compositions, of divalent manganese salts as agents for reducing the odor resulting from the decomposition by bacteria of human sweat.

The invention also relates to a process for reducing human body odors resulting from their [sic] bacterial decomposition, which consists in applying, to the axillary surface, scalp or feet, an effective amount of a divalent manganese salt.

Body odors result from the metabolization of compounds present on the skin or scalp by microorganisms of the resident body flora. The literature gives information on the main types of microorganisms responsible for the appearance of unpleasant body odors and on the main chemical families constituting these odors. Thus, diphtheroids with a lipolytic activity appear as the most frequently cited types of bacteria, with in particular Corynebacterium xerosis for axillary odors and Propionibacterium acnes for odors of the feet and scalp. With regard to the latter body region, a yeast, Pityrosporum ovale, also appears to contribute to the development of the odor.

The main families of malodorous compounds would be short chain C₃-C₁₀ fatty acids (odors of the armpits and feet), 16-androstenes (volatile C₁₇ steroids) (axillary odors), and lactones (odors of the scalp).

In the cosmetics field, it is well known to use, in topical application, products comprising active substances of antiperspirant type or of bactericide type.

Antiperspirant substances have the effect of limiting the flow of sweat. They are generally composed of aluminum salts which, first, are irritating to the skin and which, secondly, reduce the flow of sweat by altering the cutaneous physiology, which is not satisfactory.

Bactericidal substances inhibit the growth of the skin flora responsible for axillary odors. The most commonly employed bactericidal product is triclosan (2,4,4′-trichloro-2′-hydroxydiphenyl ether), which exhibits the disadvantage of significantly altering the ecology of the skin flora and of being inhibited by some compounds, such as, for example, nonionic surfactants, commonly used in the formulation of cosmetic compositions. Furthermore, the insoluble nature of triclosan in water does not allow it to be incorporated in essentially aqueous formulas.

With the aim of obtaining long term effectiveness, a search is underway for novel products which are capable of altering, of reducing and/or of removing or of preventing the development of body odors (this definition is given in the work “Cosmetic Science and Technology Series”, 1988, volume 7, chap. 10-IIIc). In addition, a search is underway for products which do not exhibit the disadvantages of the active substances used in the prior art.

U.S. Pat. No. 5,788,956 has disclosed a process for preventing or controlling cutaneous sweat flow in man, which consists in using substances [sic] P antagonists as antiperspirant agent, the antagonists including in particular manganese or magnesium salts.

This document neither discloses nor suggests the use of a divalent manganese salt for reducing the unpleasant odor resulting from the bacterial decomposition of human sweat.

Patent application WO 96/29049 has already recommended the use in cosmetics, in cleansing compositions for the skin, of polyvalent cations, including in particular manganese, as antimicrobial agents effective with regard to the pathogenic microorganism Staphylococcus aureus. However, this bacterium does not form part of the resident skin flora and does not appear as one of the main microorganisms involved in the formation of body odors.

Patent application EP-713 695 has disclosed a process for the permanent deformation of the hair, during which manganese salts have the effect in particular of neutralizing the odor of the mercaptans given off during the reduction phase. However, this document neither discloses nor suggests the use of said salts for neutralizing the odor of fatty acids, steroids and lactones which are the cause of malodorous body compounds [sic].

After much research directed at the question, the Applicant Company has now discovered, unexpectedly and surprisingly(that divalent manganese salts exhibit the property of reducing perspiration odors of the armpits and feet, and odors of the scalp, without the disadvantages of the active substances previously employed in deodorant compositions and with the advantage, for some of these compounds, of being water-soluble in advantageous and sufficient proportions to be easily capable of formulation, in particular in water-based cosmetic compositions for human topical application.

This discovery forms the basis of the present invention.

A first subject matter of the present invention is thus the use in a cosmetic composition of divalent manganese salts as agents for reducing the strength of the odor of human sweat decomposed by bacteria.

A second subject matter of the invention is a process for reducing human body odors resulting from their [sic] decomposition by bacteria, which consists in applying, to the axillary surface, feet or scalp, an effective amount of a divalent manganese salt.

A third subject matter of the invention is a cosmetic composition intended to be applied to the human skin comprising, in a cosmetically acceptable vehicle, at least one antiperspirant or deodorant active principle and, in addition, at least one divalent manganese salt as agent for reducing the strength of the odor of human sweat decomposed by bacteria, and another subject matter of the invention is also the use of said composition in or for the manufacture of antiperspirant or deodorant cosmetic products intended for human topical application.

Within the meaning of the present invention, the term “water-soluble manganese salts” is understood to mean manganese salts of organic or inorganic acids with a solubility in water, expressed as percentage by weight of Mn ion, of greater than or equal to 0.3. Water-soluble manganese salts which are particularly preferred according to the present invention are manganese chloride, manganese acetate and manganese sulfate.

Among the abovementioned salts, preference is even more particularly given to the use, according to the present invention, of manganese chloride (MnCl₂)

According to the present invention, water-soluble divalent manganese salts, in comparison with triclosan, which is well known in the field under consideration, exhibit the advantage of being more effective with regard to reducing the strength and the annoyance of the odor and of being odorless at the concentrations employed.

The divalent manganese salts according to the invention preferably represent from 0.01 to 20% by weight approximately of the total weight of the cosmetic composition, more particularly from 0.1 to 10% and more preferably still from 0.1 to 5% by weight approximately of this weight.

The deodorant active principles can be chosen, for example, from:

water-soluble zinc salts, such as, for example, zinc pyrrolidonecarboxylate (more commonly known as zinc pidolate), zinc sulfate, zinc chloride, zinc lactate, zinc gluconate and zinc phenolsulfonate; other water-insoluble zinc compounds, such as, for example, zinc ricinoleate; or bactericides, such as 2,4,4′-trichloro-2′-hydroxydiphenyl ether (triclosan) or 3,7,11-trimethyldodeca-2,5,10-trienol (farnesol).

These deodorant active principles can be present in the composition according to the invention in a proportion of approximately 0.001 to 20% by weight with respect to the total composition and preferably in a proportion of approximately 0.1 to 5% by weight.

The cosmetic composition according to the invention is formulated conventionally according to the presentation form for which it is intended.

It is more particularly formulated in a cosmetically acceptable vehicle which can in particular be essentially aqueous or can comprise organic solvents and in particular C₁-C₄ monoalcohols, preferably ethanol, to accelerate the evaporation of the product, or propylene glycol, dipropylene glycol and their ethers.

The cosmetic composition according to the invention can also be formulated as a water-in-oil or oil-in-water emulsion or as a water-in-oil-in-water triple emulsion (such emulsions are known and described, for example by C. Fox in “Cosmetics and Toiletries”, November 1986, vol. 101, pages 101-112).

The cosmetic composition of the invention can additionally comprise cosmetic adjuvants chosen from fatty substances, gelling agents, emollients, softeners, antioxidants, opacifiers, stabilizing agents, antifoaming agents, moisturizing agents, vitamins, fragrances, preservatives, surfactants, fillers, sequestering agents, polymers, propellants, basifying or acidifying agents, fragrances [sic], dyes, pigments, thickening agents or any other ingredient normally used in cosmetics for this type of application.

Of course, a person skilled in the art will take care to choose this or these possible additional compounds so that the advantageous properties intrinsically attached to the cosmetic composition according to the invention are not, or not substantially, detrimentally affected by the envisaged addition or additions.

The surfactants are preferably chosen from anionic, amphoteric or nonionic surfactants.

The fatty substances can be composed of an oil or a wax or their mixture, petroleum jelly, paraffin, lanolin, hydrogenated lanolin or acetylated lanolin; they also comprise fatty acids, fatty alcohols, such as lauryl, cetyl, myristyl, stearyl, palmityl or oleyl alcohol and 2-octyldodecanol, fatty acid esters, such as glyceryl monostearate, polyethylene glycol monostearate, isopropyl myristate, isopropyl adipate, isopropyl palmitate, octyl palmitate or benzoates of C₁₂-C₁₅ fatty alcohols (Finsolv TN from Finetex), myristyl alcohol polyoxypropylenated with 3 mol of propylene oxide (Witconol APM from Witco) or triglycerides of C₆-C₁₈ fatty acids, such as caprylic/capric acid triglycerides.

The oils are chosen from animal, vegetable, mineral or synthetic oils and in particular hydrogenated palm oil, hydrogenated castor oil, liquid petrolatum, liquid paraffin, purcellin oil (stearyl octanoate), silicone oils and isoparaffins.

The waxes are chosen from animal, fossil, vegetable, mineral or synthetic waxes. Mention may in particular be made of beeswaxes, carnauba wax, candelilla wax, sugarcane wax, japan wax, ozokerites, montan wax, microcrystalline waxes, paraffin waxes or silicone waxes and resins.

The thickening agents, preferably nonionic thickening agents, can be chosen from modified or unmodified guar gums and celluloses, such as hydroxypropylated guar gum or cetylhydroxyethylcellulose, silicas, such as, for example, Bentone Gel MiO, sold by NL Industries, or Veegum Ultra, sold by Polyplastic.

The cosmetic composition can comprise emollients, which contribute to a soft, dry and nonsticky feeling on application of the composition to the skin. These emollients can be chosen from products of the volatile silicone type, nonvolatile silicones and other nonvolatile emollients.

The volatile silicones are defined in a known way as compounds which are volatile at ambient temperature. Mention may be made, among these compounds, of cyclic and linear volatile silicones of the dimethylsiloxane type, the chains of which comprise from 3 to 9 silicone residues. Cyclomethicone D4 or cyclomethicone D5 is preferably chosen.

The nonvolatile silicones are defined in a known way as compounds with a low vapor pressure at ambient temperature. These compounds include: polyalkyl-siloxanes, in particular linear polyalkylsiloxanes, such as, for example, linear polydimethylsiloxanes or dimethicones sold by Dow Corning under the name of “Dow Corning 200 Fluid”; polyalkylarylsiloxanes, such as, for example, the polymethylphenylsiloxanes sold by Dow Corning under the name of “Dow Corning 556 Fluid”; and polyether and siloxane copolymers, such as, for example, dimethicone copolyols.

Mention may be made, among the nonvolatile emollients which can be used in the present invention, of, for example: hydrocarbonaceous derivatives, mineral oils, fatty alcohols, esters of C₃-C₁₈ alcohols with C₃-C₁₈ acids, esters of benzoic acid with C₁₂-C₈ alcohols and their mixtures, C₂-C₆ polyols preferably chosen from glycerol, propylene glycol or sorbitol, or polyalkylene glycol polymers.

The amounts of these various constituents which can be present in the cosmetic composition according to the invention are those conventionally used for the presentation forms under consideration.

The composition according to the invention can thus be provided in the form of a lotion, cream or fluid gel distributed as an aerosol spray, as a pump-action spray or as a roll-on, in the form of a thick cream distributed as a tube and in the form of a stick or powder, and can comprise, in this respect, the ingredients and propellants generally used in products of this type and which are well known to a person skilled in the art.

It can also constitute woven or nonwoven and natural, synthetic or artificial fiber supports intended for topical application (more commonly referred to as “wipes”).

Concrete but in no way limiting examples illustrating the invention will now be given.

### EXAMPLE 1

### In vitro Test of Evaluation of Odors

Comparison of the effectiveness of manganese chloride [(MnCl₂) (invention)] versus that of the deodorant active principle of the prior art (triclosan).

A test of inhibition of the evolution of odors was carried out on natural sweat.

The principle of this test consists in adding the deodorant agent to fresh sweat and then in carrying out an olfactory evaluation by a jury of experts after incubating for 18 hours and then 24 hours at 37° C.

Thus, the axillary sweat was taken from several human models in a sauna and was combined to produce a sample of sweat. In order to obtain the characteristic nauseating odor of sweat, this sample of sweat was incubated for 18 hours and then 24 hours at a temperature of 37° C. before introducing the active substance. 1 ml of the sample of incubated sweat was introduced into each flask of active substance to be tested and into a control flask (without active substance). The active substance was then introduced into each of the flasks to be tested.

Results:

The results were given as percentages of the variation (reduction) in the odor and in the hedonic grade with respect to the control sweat without deodorant agent. Per 1 mg of active material per ml of sweat, i.e. 0.1%:

| | Mean % of reduction in | Mean % of reduction in |
|---|---|---|
| Deodorant | the strength of the odor | the odor annoyance |
| agent | (mean over 18 h and 24 h) | (mean over 18 h and 24 h) |
| MnCl₂ | −43 | −31 |
| Triclosan | −39 | −27 |

The results show that manganese chloride exhibits a reduction with regard to the strength and the annoyance of the odor of natural sweat for at least 18 h which is as great as [lacuna] triclosan, indeed even greater.

### EXAMPLE 2

### Determination of the Biocidal Activity

The microbiostatic activity of MnCl₂ was determined in comparison with triclosan (deodorant active principle of the prior art) by the method of dilutions in agar medium.

Each of the 2 compounds was presented in the form of an ethanolic solution, MnCl₂ at 10% of active material (AM) and triclosan at 1% AM.

One hundred μl of the ethanolic solution were added to 0.9 ml of agar culture heated beyond the melting point to 45° C. (medium chosen as a function of the nutrient requirements of the strain studied). After stirring with a Vortex device, successive dilutions in geometric progression in a ratio of 2 of the solution obtained were carried out using agar culture medium heated beyond the melting point to 45° C. One ml of each dilution was introduced into the wells of a microplate (Flacon [sic], 24 wells). Four μl of microbial suspension were deposited at the surface of the agar medium.

Two control wells were also prepared: 100 μl H₂O+0.9 ml of culture medium (growth control), 100 μl ethanol+0.9 ml of culture medium.

After incubating under conditions (temperature, atmosphere and duration) dependent on the strain studied, the Minimum Inhibitory Concentration (MIC) is given by the lowest concentration of product which inhibits microbial growth.

Results: expressed in % w/v

For the control wells (H₂O or ethanol), the observation of microbial growth made it possible to exclude the presence of a placebo effect.

| | MnCl₂ | Triclosan |
|---|---|---|
| Staphylococcus epidermidis | 0.23 | 3 × 10⁻⁸ |
| Corynebacterium xerosis | 0.23 | 4 × 10⁻⁶ |

The results show that the biocidal activity of MnCl₂ is very markedly less than that of triclosan with regard to the 2 bacterial strains tested of the resident skin flora and that the effectiveness of MnCl₂ does not appear to be directly related to a biocidal effect since it is found at concentrations 2 times lower than the MIC.

### EXAMPLE 3

### Spray (Pump-Action Spray)

| MnCl₂.4H₂O | 3.00 | g |
|---|---|---|
| Fragrance, dyes | q.s. | |
| Ethyl alcohol, 95 vol %, denatured | 100 | g |

### EXAMPLE 4

### Water-In-Silicone Emulsion

| Silicone DC 245 Fluid (Dow Corning) | | 6.60 | g |
|---|---|---|---|
| Silicone DC 5225 C (Dow Corning) | | 9.40 | g |
| Ethyl alcohol | | 11.00 | g |
| Propylene glycol | | 37.00 | g |
| MnCl₂.4H₂O | | 3.00 | g |
| Fragrance, preservatives, dyes | | q.s. | |
| Demineralized water | q.s. for | 100 | g |

### EXAMPLE 5

### Transparent Aqueous/Alcoholic Roll-On

| Oxyethylenated (20 EO) and oxypropylenated | | 2.00 | g |
|---|---|---|---|
| (5 PO) cetyl alcohol | | | |
| Hydroxyethylcellulose | | 0.75 | g |
| MnCl₂.4H₂O | | 2.50 | g |
| Ethyl alcohol, 95 vol %, denatured | | 45.00 | g |
| Fragrance | | q.s. | |
| Demineralized water | q.s. for | 100 | g |

### EXAMPLE 6

### Cream for the Feet

| Glyceryl mono/distearate/polyethylene | | 8.00 | g |
|---|---|---|---|
| glycol (100 EO) stearate mixture | | | |
| Cetyl alcohol | | 2.50 | g |
| Microcrystalline wax/beeswax/ | | 1.00 | g |
| polyethylene mixture | | | |
| Liquid jojoba wax | | 6.00 | g |
| Oxyethylenated (3 EO) myristyl myristate | | 2.00 | g |
| MnCl₂.4H₂O | | 5.00 | g |
| Fragrance, preservatives | | q.s. | |
| Demineralized water | q.s. for | 100 | g |

### EXAMPLE 7

### Lotion for the Scalp

| Ethyl alcohol, 95 vol %, denatured | | 55.00 | g |
|---|---|---|---|
| MnCl₂.4H₂O | | 3.00 | g |
| Propylene glycol | | 10.00 | g |
| Fragrance, dye | | q.s. | |
| Demineralized water | q.s. for | 100 | g |

1. Use in a cosmetic composition of at least one divalent manganese salt as agent for reducing the strength of the odor of human sweat decomposed by bacteria.

2. The use as claimed in claim 1, characterized in that the manganese salt is a water-soluble salt.

3. The use as claimed in claims 1 and 2, characterized in that the salt is manganese chloride (MnCl₂).

4. The use as claimed in any one of the preceding claims, characterized in that the divalent manganese salt or salts represent from 0.01% to 20% of Active Material by weight with respect to the total weight of the composition.

5. The use as claimed in claim 4, characterized in that the divalent manganese salt or salts represent from 0.1 to 10% of Active Material by weight with respect to the total weight of the composition.

6. The use as claimed in claim 5, characterized in that the divalent manganese salt or salts represent from 0.1 to 5% of Active Material by weight with respect to the total weight of the composition.

7. A cosmetic composition intended to be applied to human skin comprising, in a cosmetically acceptable vehicle, at least one antiperspirant or deodorant active principle, characterized in that it additionally comprises at least one divalent manganese salt as agent for reducing the strength of the odor of human sweat decomposed by bacteria.

8. The composition as claimed in claim 7, characterized in that the vehicle is essentially aqueous.

9. The composition as claimed in claim 8, characterized in that the vehicle additionally comprises organic solvents.

10. The composition as claimed in any one of claims 7 to 9, characterized in that it additionally comprises cosmetic adjuvants chosen from fatty substances, gelling agents, emollients, softeners, antioxidants, opacifiers, stabilizing agents, antifoaming agents, moisturizing agents, vitamins, fragrances, preservatives, surfactants, fillers, sequestering agents, polymers, propellants, basifying or acidifying agents, fragrances [sic], dyes, pigments, thickening agents or any other ingredient normally used in cosmetics.

11. The composition as claimed in any one of claims 7 to 10, characterized in that it is formulated as a water-in-oil or oil-in-water emulsion or as a water-in-oil-in-water triple emulsion.

12. The composition as claimed in any one of claims 7 to 11, characterized in that it is provided in the form of a lotion, cream or fluid gel distributed as an aerosol spray, as a pump-action spray or as a roll-on, in the form of a thick cream distributed as a tube and in the form of a stick or powder, or of woven or nonwoven and natural, synthetic or artificial fiber supports.

13. Use of the composition as claimed in any one of claims 7 to 12, as or for the manufacture of antiperspirant or deodorant cosmetic products intended for human topical application.

14. A process for reducing human body odors resulting from their [sic] decomposition by bacteria, which consists in applying, to the axillary surface, scalp or feet, an effective amount of a divalent manganese salt.

15. The process as claimed in claim 14, characterized in that manganese chloride (MnCl₂) is concerned.
